(19) United States
(12) Patent Application Publication
(10) Pub. No.: US 2020/0289852 A1
(43) Pub. Date: Sep. 17, 2020

(54) METHOD AND APPARATUS FOR CONTROLLING RADIOTHERAPY DEVICE, AND RADIOTHERAPY SYSTEM

(71) Applicant: OUR UNITED CORPORATION, Xi'an (CN)

(72) Inventor: Jinsheng LI, Xi'an (CN)

(21) Appl. No.: 16/633,788

(22) PCT Filed: Jul. 31, 2017
(86) PCT No.: PCT/CN2017/095323

§ 371 Date: Jan. 24, 2020

(51) Int. Cl. A61N 5/10

(52) U.S. Cl. CPC A61N 5/1067; A61N 5/1049; A61N 5/1047

(57) ABSTRACT

An apparatus for controlling a radiation therapy device includes a drive module, a control module and an input module. The drive module and the control module are connected with the input module, respectively. The input module is configured to acquire a first arc-shaped trajectory. The drive module is configured to drive a radiation therapy head to move along the first arc-shaped trajectory at a first speed. The control module is configured to control the radiation therapy head to emit rays and form a radiation field, the radiation field includes at least one sub-radiation field, and the first speed is smaller than a preset speed threshold. The radiation therapy head performs a circumferential movement. A circumferential trajectory along which the radiation therapy head moves includes at least two first arc-shaped trajectories, and any two first arc-shaped trajectories are discontinuous.

### CROSS-REFERENCE TO RELATED APPLICATION

This application is a 371 of PCT international patent application No.: PCT/CN2017/095323 filed on Jul. 31, 2017.

### TECHNICAL FIELD

The present disclosure relates to the field of medical technology, and particularly to a control method and apparatus for a radiation therapy device, and a radiation therapy system.

### BACKGROUND

In modern medicine, radiation therapy is an important means for treating malignant tumors. The radiation therapy refers to the use of high-energy radioactive rays to kill tumors. At present, a radiation therapy head is mainly used for performing the radiation therapy. Generally, the radiation therapy head includes a ray source and a radiation field collimation system, and a multi-leaf collimator is a part of the radiation field collimation system. For example, the ray source may be an accelerator. The accelerator is configured to emit X rays, and the multi-leaf collimator is configured to generate a satisfactory radiation field. The radiation field refers to an area and a shape which are irradiated by the X rays, and defines a ray irradiation range. The X rays emitted from the accelerator are irradiated to a tumor lesion area through the radiation field generated by the multi-leaf collimator. In this process, the X rays are further irradiated to normal tissue and organ around the tumor lesion area.

In the volumetric modulated arc therapy (VMAT) technology, the radiation therapy head may employ the X rays to rotatably irradiate the tumor lesion area within a 360-degree angle. In order to meet the requirement for radiation dose distribution, that is, in order to meet the requirement for higher radiation dose in the tumor lesion area and lower radiation dose in the normal tissue and organ around the tumor lesion area, the accelerator continuously emits the X rays during rotation of the radiation therapy head. The shape and the size of the radiation field generated by the multi-leaf collimator constantly change.

In a process of implementing the present disclosure, the inventors found that the above-mentioned technology at least has the following problems.

Since using the X rays to rotatably irradiate the tumor lesion area within the 360-degree angle, the radiation therapy head cannot perform directional treatment at a fixed angle and thus a movement trajectory may not meet the requirement for multi-directional treatment.

### SUMMARY

Embodiments of the present disclosure provide a method and apparatus for controlling a radiation therapy device, and a radiation therapy system. The technical solutions are described as below.

According to a first aspect of the embodiments of the present disclosure, there is provided an apparatus for controlling a radiation therapy device, including a drive module, a control module and an input module, the drive module and the control module are connected with the input module, respectively,

the input module is configured to acquire a first arc-shaped trajectory, and the first arc-shaped trajectory is determined according to a medical image of a patient;

the drive module is configured to drive a radiation therapy head to move along the first arc-shaped trajectory at a first speed, and the first speed is smaller than a preset speed threshold;

the control module is configured to control the radiation therapy head to emit rays and form a radiation field when the radiation therapy head moves along the first arc-shaped trajectory, wherein the radiation field includes at least one sub-radiation field; and

the radiation therapy head performs a circumferential movement, and a circumferential trajectory along which the radiation therapy head moves includes at least two first arc-shaped trajectories, and any two first arc-shaped trajectories are discontinuous.

According to a second aspect of the embodiments of the present disclosure, there is provided a method for controlling a radiation therapy device, including:

acquiring a first arc-shaped trajectory, wherein the first arc-shaped trajectory is determined according to a medical image of a patient; and

driving a radiation therapy head to move along the first arc-shaped trajectory at a first speed to control the radiation therapy head to emit rays and form a radiation field, wherein the radiation field includes at least one sub-radiation field, and the first speed is smaller than a preset speed threshold;

wherein the radiation therapy head performs a circumferential movement, and a circumferential trajectory along which the radiation therapy head moves includes at least two first arc-shaped trajectories, and any two first arc-shaped trajectories are discontinuous.

According to a third aspect of the embodiments of the present disclosure, there is provided a radiation therapy system, including a controller and a. radiation therapy head,

the controller includes a control apparatus for a radiation therapy device according to any one of the first aspect.

According to a fourth aspect of the embodiments of the present disclosure, there is provided a control apparatus for a radiation therapy device, including:

a processor; and

a memory, which is configured to store an executable instruction of the processor;

wherein the processor is configured to:

acquire a first arc-shaped trajectory;

drive the radiation therapy head to move along the first arc-shaped trajectory at a first speed, and control the radiation therapy head to emit rays and form a radiation field, wherein the radiation field includes at least one sub-radiation field, and the first speed is smaller than a preset speed threshold;

wherein the radiation therapy head performs a circumferential movement, and a circumferential trajectory along which the radiation therapy head moves includes at least two first arc-shaped trajectories, and any two first arc-shaped trajectories are discontinuous.

According to a fifth aspect of the embodiments of the present disclosure, there is provided a storage medium. The storage medium stores an instruction, which is configured to: when the storage medium is run on a terminal, cause the terminal to perform the control method for the radiation therapy device according to the second aspect.

According to a sixth aspect of the embodiments of the present disclosure, there is provided a terminal program product including an instruction, which is configured to: when the terminal program product is run on a terminal, cause the terminal to perform the control method for the radiation therapy device according to the second aspect.

The technical solutions provided by the embodiments of the present disclosure have the following beneficial effects.

According to the method and apparatus for controlling the radiation therapy device, and the radiation therapy system provided by the embodiments of the present disclosure, the radiation therapy head can be controlled to emit the rays and form the radiation field when moving along the first arc-shaped trajectory at the first speed, wherein the radiation field includes at least one sub-radiation field, and the first speed is less than the preset speed threshold. The radiation therapy head performs the circumferential movement, and the circumferential trajectory along which the radiation therapy head moves includes at least two first arc-shaped trajectories, and any two first arc-shaped trajectories are discontinuous. The first arc-shaped trajectory may be set according to the treatment need to meet special needs of clinical treatment plans for different treatments. For example, the first arc-shaped trajectory may be a movement trajectory which avoids an important or sensitive tissue and organ around the tumor during irradiation of radioactive rays, so that damage to normal tissues or organs during radiation therapy may be reduced. In addition, when the radiation therapy head moves along the first arc-shaped trajectory, the radiation field in each direction includes at least one sub-radiation field, so that the use flexibility of the radiation therapy head is improved, and a larger possibility for improving the quality of the treatment plan and the treatment effect is provided.

### BRIEF DESCRIPTION OF THE DRAWINGS

In order to describe the technical solutions in the embodiments of the present more clearly, the following briefly introduces the accompanying drawings required for describing the embodiments. Apparently, the accompanying drawings in the following description show merely some embodiments of the present disclosure, and a person of ordinary skill in the art may also derive other drawings from these accompanying drawings without creative efforts.

FIG. 1 is a schematic diagram of a radiation therapy system according to an embodiment of the present disclosure;

FIG. 2-1 is a schematic diagram showing a structure of a control apparatus for a radiation therapy device according to an embodiment of the present disclosure;

FIG. 2-2 is a schematic diagram of a circumferential trajectory along which a radiation therapy head moves according to an embodiment of the present disclosure;

FIG. 2-3 is a schematic diagram of a CT image of an abdominal cavity of a patient according to an embodiment of the present disclosure;

FIG. 2-4 is a schematic diagram of a rectangular radiation field according to an embodiment of the present disclosure;

FIG. 2-5 is a schematic diagram of a radiation therapy head according to an embodiment of the present disclosure;

FIG. 3-1 is a flow chart of a control method for a radiation therapy device according to an embodiment of the present disclosure;

FIG. 3-2 is a flow chart of an another control method for a radiation therapy device according to an embodiment of the present disclosure;

FIG. 4 is a schematic diagram showing a structure of a radiation therapy system according to an embodiment of the present disclosure;

FIG. 5 is a block diagram of a control apparatus for another radiation therapy device according to an embodiment of the present disclosure.

### DETAILED DESCRIPTION

In order to describe the objects, technical solutions and advantages in the embodiments of the present more clearly, the present disclosure will be described in detail below in combination with the accompanying drawings. Apparently, the described embodiments are merely some embodiments, rather than all embodiments, of the present disclosure. Based on the embodiments of the present disclosure, all other embodiments derived by a person of ordinary skill in the art without creative efforts shall fall within the protection scope of the present disclosure.

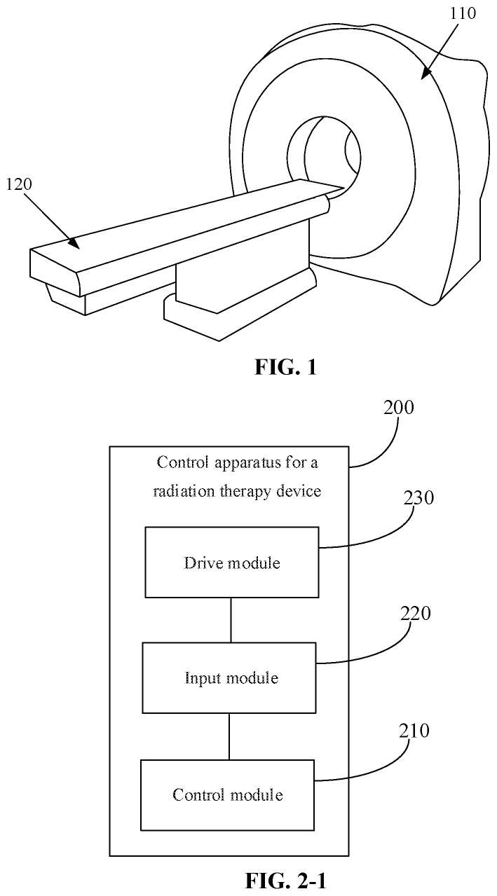

Reference is made to FIG. 1, which shows a schematic diagram of a radiation therapy system according to an embodiment of the present disclosure. The radiation therapy system at least includes a frame 110, a treatment couch 120 and a controller (not shown in FIG. 1). Generally, the frame 110 is a drum-type frame, and a radiation therapy head (not shown in FIG. 1) is disposed on the frame 110. The controller controls the frame 110 to rotate. The radiation therapy head can rotate with the frame 110. The radiation therapy head is controlled by a controller to emit rays and form a radiation field. The controller controls the treatment couch 120 to move, so that the rays emitted from the radiation therapy head is irradiated to a tumor lesion area of a patient.

It should be noted that the present disclosure is exemplified only by using the frame as the drum-type frame. For example, the frame may further be a C-shaped arm, a cantilever type arm, a semi-arc-shaped arm, or the like. The radiation therapy head will not be particularly limited in the present disclosure. For example, the radiation therapy head may employ cobalt-60 as a ray source conformal treatment head, or may further be an accelerator conformal treatment head, a neutron conformal treatment head or a proton conformal treatment head, and the like. Generally, the conformal treatment head includes a ray source and a multi-leaf collimator.

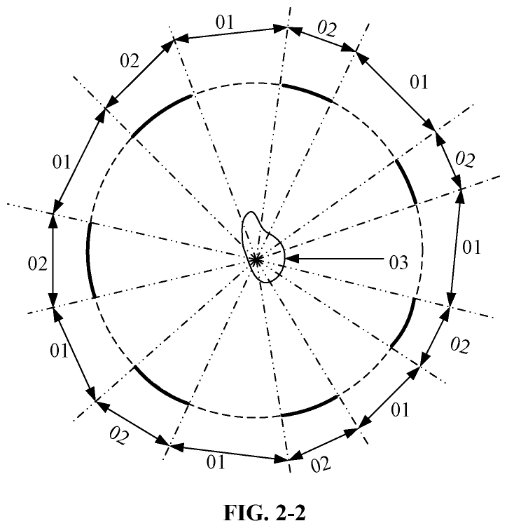

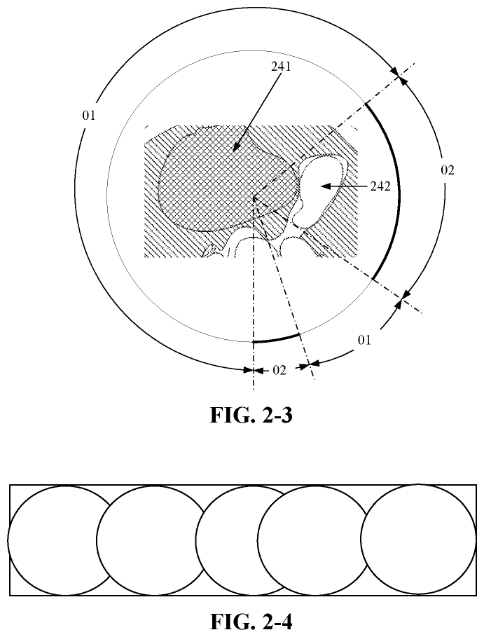

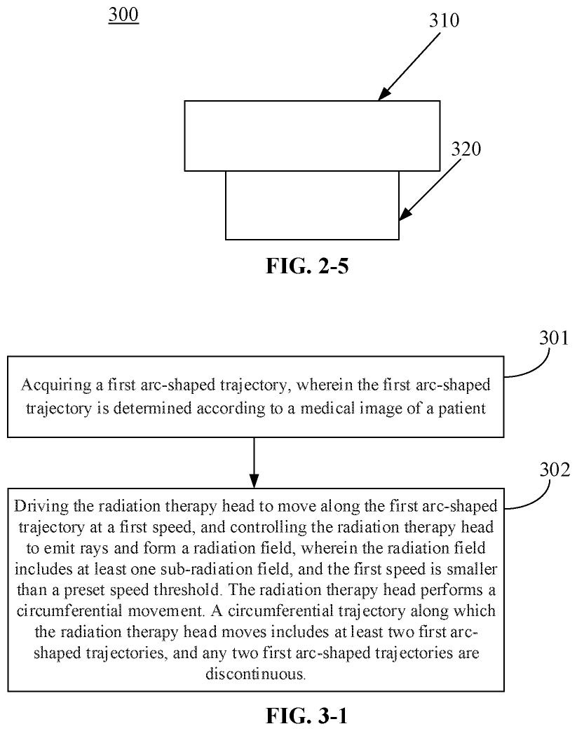

Reference is made to FIG. 2-1, which shows a schematic diagram showing a structure of a control apparatus 200 for a radiation therapy device according to an embodiment of the present disclosure. The control apparatus 200 for the radiation therapy device includes a drive module 230, a control module 210 and an input module 220, wherein the drive module 230 and the control module 210 are connected with the input module 220, respectively.

The input module 220 is configured to acquire a first arc-shaped trajectory, wherein the first arc-shaped trajectory is determined according to a medical image of a patient.

In an actual application, a location of a tumor lesion area may be determined according to the medical image of the patient, and then a treatment plan is formulated according to the location of the tumor lesion area. The treatment plan includes the first arc-shaped trajectory. For example, the first arc-shaped trajectory may be a movement trajectory which avoids an important or sensitive tissue and organ around the tumor during irradiation of radioactive rays, so that the damage to normal tissue and organ during radiation therapy may be reduced. The treatment plan may further include data, such as a radiation therapy cycle, duration of each radiation therapy, a radiation dose, and a conformal shape of an irradiation target area.

The drive module 230 is configured to drive the radiation therapy head to move along the first arc-shaped trajectory at a first speed, wherein the first speed is smaller than a preset speed threshold.

The control module 210 is configured to control the radiation therapy head to emit rays and form a radiation field when the radiation therapy head moves along the first arc-shaped trajectory. The radiation field includes at least one sub-radiation field.

The radiation therapy head performs a circumferential movement, as shown in FIG. 2-2. A circumferential trajectory along which the radiation therapy head moves includes at least two first arc-shaped trajectories 01, and any two first arc-shaped trajectories 01 are discontinuous. The tumor is represented by 03 in FIG. 2-2. FIG. 2-2 exemplarily shows seven first arc-shaped trajectories 01.

In summary, according to the control apparatus for the radiation therapy device provided by the embodiment of the present disclosure, the control module can control the radiation therapy head to emit the rays and form the radiation field when the radiation therapy head moves along the first arc-shaped trajectory at the first speed, wherein the radiation field includes at least one sub-radiation field, and the first speed is less than the preset speed threshold. The radiation therapy head performs the circumferential movement, and the circumferential trajectory along which the radiation therapy head moves includes at least two first arc-shaped trajectories, and any two first arc-shaped trajectories are discontinuous. The first arc-shaped trajectory may be set according to the treatment need to meet special needs of clinical treatment plans for different treatments. In addition, when the radiation therapy head moves along the first arc-shaped trajectory, the radiation field formed in each direction includes at least one sub-radiation field, so that the use flexibility of the radiation therapy head can be improved.

For example, the preset speed threshold ranges from 2 minutes per revolution to 6 minutes per revolution, that is, the time taken for each revolution of the radiation therapy head ranges from 2 minutes to 6 minutes. For instance, the first speed is 15 minutes per revolution, which is less than the preset speed threshold.

Further, as shown in FIG. 2-2, the circumferential trajectory along which the radiation therapy head moves further includes a second arc-shaped trajectory 02. The first arc-shaped frame 01 and the second arc-shaped frame 02 are alternately arranged. The number of the second arc-shaped trajectories 02 and the number of the first arc-shaped trajectories 01 may be the same or different. FIG. 2-2 exemplarily shows seven second arc-shaped trajectories 02. The second arc-shaped trajectory may be determined according to the location of the tumor lesion area. The treatment plan may further include the second arc-shaped trajectory.

The drive module 230 in FIG. 2-1 is further configured to drive the radiation therapy head to move along the second arc-shaped trajectory at a second speed. The control module 210 is further configured to control the radiation therapy head to emit rays and form the radiation field when the radiation therapy head moves along the second arc-shaped trajectory 02 at the second speed. The radiation field includes at least one sub-radiation field, and the second speed is greater than or equal to a preset speed threshold, and the preset speed threshold ranges from 2 minutes per revolution to 6 min per revolution. For example, the second speed may be 6 minutes per revolution, that is, the time taken for each revolution of the radiation therapy head is 6 minutes.

It should be noted that, when the radiation therapy head moves along the second arc-shaped trajectory at the second speed, the speed of the radiation therapy head is variable.

Alternatively, the radiation field formed by the radiation therapy head includes a plurality of sub-radiation fields.

In the embodiment of the present disclosure, when the radiation therapy head moves along the second arc-shaped trajectory, the movement speed of the radiation therapy head is relatively high, which accelerates the treatment speed of the entire process and shortens the treatment time. Moreover, the radiation field formed by the radiation therapy head in each direction includes a plurality of sub-radiation fields, and the use flexibility of the radiation therapy head is relatively high.

Further, the control apparatus for the radiation therapy device further includes a display module, which is configured to display an irradiated tumor of the patient in real time. A staff member may make related treatment suggestions from a display result of the display module.

Particularly, the input module 220 is configured to acquire the medical image of the patient and determine the location of the tumor lesion area according to the medical image; and determine the first arc-shaped trajectory according to the location of the tumor lesion area. The input module 220 may formulate the treatment plan according to the location of the tumor lesion area, and the treatment plan includes the first arc-shaped trajectory. In addition, it is possible for the staff member to determine the location of the tumor lesion area according to the medical image, and then to formulate the treatment plan including the first arc-shaped trajectory according to the location of the tumor lesion area. A manner of determining the first arc-shaped trajectory will not be limited in the embodiment of the present disclosure.

Further, the input module 220 is further configured to determine the location of the designated tissue and organ around the tumor lesion area according to the medical image, and determine the first arc-shaped trajectory according to the location of the designated tissue and organ around the tumor lesion area. The designated tissue and organ may be an important or sensitive tissue and organ at risk. There may be one or more important or sensitive tissues and organs.

The medical image may be a patient image formed by computed tomography (CT), nuclear magnetic resonance, positron emission tomography (PET), PET-CT or B-ultrasound.

In the embodiment of the present disclosure, the input module may determine the first arc-shaped trajectory according to the location of the tumor lesion area, and may further determine the first arc-shaped trajectory in combination with the location of the tumor lesion area and the location of the designated tissue and organ around the tumor lesion area. The process of determining the first arc-shaped trajectory by the input module may refer to a manner, which is adopted by the intensity-modulated radiation therapy (IMRT) technology, for determining a frame angle. In the IMRT technology, when the radiation therapy head is rotated to a location with a predetermined frame angle, the radiation therapy head remains stationary and then emits rays.

In the embodiment of the present disclosure, the first arc-shaped trajectory corresponds to the tumor lesion area, and the second arc-shaped trajectory may correspond to the designated tissue and organ around the tumor lesion area. When the radiation therapy head moves along the first arc-shaped trajectory, a movement speed of the radiation therapy head decreases, and the radiation therapy head forms a radiation field including a plurality of sub-radiation fields in each direction, so that the tumor lesion area obtains a relatively high radiation dose. When the radiation therapy head moves along the second arc-shaped trajectory, the movement speed of the radiation therapy head increases, so that the important or sensitive tissue and organ at risk obtains a relatively low radiation dose. Accordingly, the damage of radiation to the normal tissue and organ is reduced, and the treatment effect is improved.

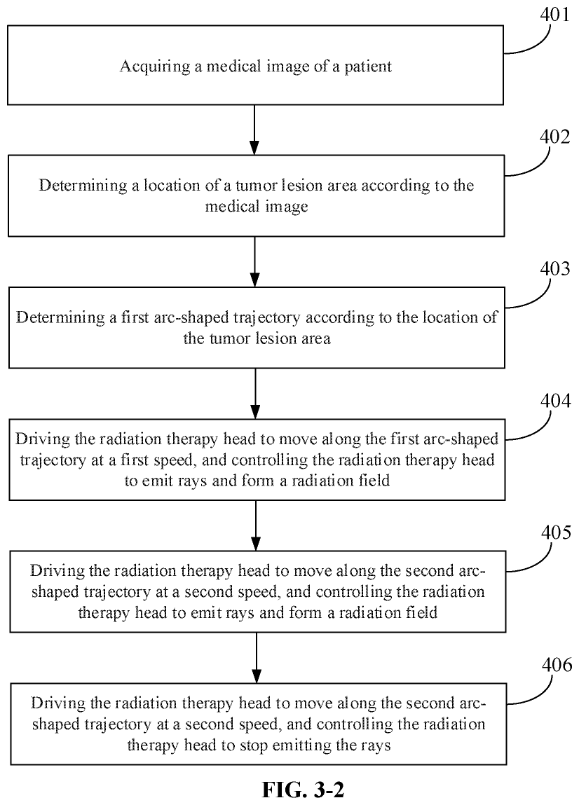

For example, FIG. 2-3 shows a schematic diagram of a CT image of an abdominal cavity of a patient. In FIG. 2-3, the tumor lesion area is represented by 241 and the sensitive tissue and organ at risk is represented by 242. The first arc-shaped trajectory 01 which is determined according to the locations of the tumor lesion area 241 and the sensitive tissue and organ 242 may be shown in FIG. 2-3, and the second arc-shaped trajectory is represented by 02. Since the first arc-shaped trajectory 01 avoids the sensitive tissue and organ 242, the radiation dose Obtained by the sensitive tissue and organ 242 is relatively low. Accordingly, the degree of damage to the sensitive tissue and organ 242 is relatively low.

Alternatively, the radiation field formed by the radiation therapy head includes a plurality of sub-radiation fields, and sizes and shapes of the plurality of sub-radiation fields included in the radiation field formed by the radiation therapy head are different from each other. When the radiation therapy head moves along the first arc-shaped trajectory or the second arc-shaped trajectory, the radiation field in each direction includes a plurality of sub-radiation fields, and sizes and shapes of the plurality of sub-radiation fields are different from each other. The amount of the rays can be effectively adjusted by the plurality of sub-radiation fields. Therefore, a treatment process has a relatively large space for optimization, which further reduces the amount of the rays passing through the important or sensitive tissue and organ at risk and reduces the radiation dose obtained; and increases the amount of the rays passing through the tumor lesion area and increases the radiation dose obtained, so that the requirement for radiation dose distribution can be effectively met, and the treatment effect is improved.

In addition, the sizes and the shapes of the plurality of sub-radiation fields included in the radiation field may be the same. That is, the radiation field includes a plurality of same sub-radiation fields. FIG. 2-4 shows a schematic diagram of a rectangular radiation field, wherein the rectangular radiation field includes a plurality of same circular sub-radiation fields.

The present disclosure is described by using an example in which the radiation field formed by the radiation therapy head includes the plurality of sub-radiation fields. Certainly, the radiation field formed by the radiation therapy head may include one sub-radiation field. For example, when the radiation therapy head moves along the first arc-shaped trajectory, one radiation field is always maintained until the radiation therapy head moves along the second arc-shaped trajectory, another radiation field starts to be formed, the radiation field formed when the radiation therapy head moves along the first arc-shaped trajectory and the radiation field formed when the radiation therapy head moves along the second arc-shaped trajectory may be the same or different.

In an implementation, the control module 210 is configured to control the radiation therapy head to continuously emit rays and sequentially form a plurality of sub-radiation fields.

In such an implementation, with reference to FIG. 2-2 and FIG. 2-4, the radiation therapy head can continuously emit the rays and sequentially form a plurality of sub-radiation fields when moving along the first arc-shaped trajectory or the second arc-shaped trajectory. Sizes and shapes of the plurality of sub-radiation fields may be different or the same. In a practical application, the sizes and the shapes of the plurality of sub-radiation fields are different. The radiation therapy head may adjust the intensity of the rays emitted within a unit time. Particularly, the intensity of the rays may be adjusted according to actual needs under the control of a controller.

In another implementation, the control module 210 is configured to control the radiation therapy head to emit rays every time one sub-radiation field is formed.

In such an implementation, with reference to FIG. 2-2 and FIG. 2-4, when the radiation therapy head moves along the first arc-shaped trajectory or the second arc-shaped trajectory, the radiation therapy head does not continuously emit rays, but emits the rays after one sub-radiation field is formed, and stops emitting the rays after an irradiation operation is completed; and then, emits the rays after another sub-radiation field is formed, and stops emitting the rays after the irradiation operation is completed. After that, the process is executed cyclically until the irradiation operation of the first arc-shaped trajectory or the second arc-shaped trajectory is completed. The sizes and the shapes of the plurality of sub-radiation fields may be different or the same. Preferably, the sizes and the shapes of the plurality of sub-radiation fields are different. The radiation therapy head may adjust the intensity of the rays emitted within a unit time. Particularly, the intensity of the rays may be adjusted according to actual needs under the control of the controller.

Further, the control module 210 is configured to control the radiation therapy head to emit rays with an intensity less than a preset intensity threshold when the radiation therapy head moves along the second arc-shaped trajectory.

Alternatively, the control module 210 is further configured to control the radiation therapy head to stop emitting the rays when the radiation therapy head moves along the second arc-shaped trajectory.

By reducing the intensity of the rays or controlling the radiation therapy head to stop emitting the rays, the amount of the rays passing through the important or sensitive tissue and organ at risk is further reduced. Accordingly, the radiation dose obtained is relatively low.

In the embodiment of the present disclosure, at least two of central angles corresponding to all the first arc-shaped trajectories included in the circumferential trajectory along which the radiation therapy head moves are different. For example, the central angles of the seven first arc-shaped trajectories 01 in FIG. 2-2 may be different, and may be particularly determined according to the location of the tumor lesion area and the location of the designated tissue and organ around the tumor lesion area, which will not be limited in the embodiment of the present disclosure.

As shown in FIG. 2-5, a treatment head 300 includes a ray source 310 and a multi-leaf collimator 320. Both the ray source and the multi-leaf collimator are connected with the control module.

The control module is configured to control the ray source 310 to emit rays, and control the multi-leaf collimator 320 to form at least one sub-radiation field.

For example, the ray source may be an accelerator or an isotope ray source. The accelerator is configured to emit X rays, and the isotope ray source may be for example Co-60 for emitting gamma rays.

In summary, according to the control apparatus for the radiation therapy device provided by the embodiment of the present disclosure, the control module can control the radiation therapy head to emit the rays and form the radiation field when the radiation therapy head moves along the first arc-shaped trajectory at the first speed, wherein the radiation field includes at least one sub-radiation field, and the first speed is less than the preset speed threshold. The radiation therapy head performs the circumferential movement, and the circumferential trajectory along which the radiation therapy head moves includes at least two first arc-shaped trajectories, and any two first arc-shaped trajectories are discontinuous. The radiation therapy head may avoid the important or sensitive tissue and organ when moving along the first arc-shaped trajectory, thereby preventing the important or sensitive tissue and organ from being damaged. The control apparatus can meet the requirement for radiation dose distribution, such that the treatment plant has a relatively large space for optimization. A better treatment plant for treatment may be generated, and a larger possibility for improving the quality of the treatment plan and the treatment effect is provided. In addition, when the radiation therapy head moves along the first arc-shaped trajectory, the radiation field formed in each direction includes at least one sub-radiation field, so that the use flexibility of the radiation therapy head is improved, and a larger possibility for improving the quality of the treatment plan and the treatment effect is provided.

Reference is made to FIG. 3-1, which shows a flow chart of a control method for a radiation therapy device according to an embodiment of the present disclosure. The control method includes the following steps.

In step 301, a first arc-shaped trajectory is acquired, wherein the first arc-shaped trajectory is determined according to a medical image of a patient.

In an actual application, a location of a tumor lesion area may be determined according to the medical image of the patient, and then a treatment plan is formulated according to the location of the tumor lesion area. The treatment plan includes the first arc-shaped trajectory. For example, the first arc-shaped trajectory may be a movement trajectory which avoids an important or sensitive tissue and organ around the tumor during irradiation of radioactive rays, so that the damage to normal tissue and organ during radiation therapy may be reduced. The treatment plan may further include data, such as a radiation therapy cycle, duration of each radiation therapy, a radiation dose, and a conformal shape of an irradiation target area.

In step 302, the radiation therapy head is driven to move along the first arc-shaped trajectory at a first speed, and the radiation therapy head is controlled to emit rays and form a radiation field, wherein the radiation field includes at least one sub-radiation field, and the first speed is smaller than a preset speed threshold. The radiation therapy head performs a circumferential movement. A circumferential trajectory along which the radiation therapy head moves includes at least two first arc-shaped trajectories, and any two first arc-shaped trajectories are discontinuous.

In summary, according to the control method of the radiation therapy device provided by an embodiment of the present disclosure, the radiation therapy head can be controlled to emit the rays and form the radiation field when the radiation therapy head moves along the first arc-shaped trajectory at the first speed, wherein the radiation field includes at least one sub-radiation field, and the first speed is smaller than a preset speed threshold. The radiation therapy head performs the circumferential movement, and the circumferential trajectory along which the radiation therapy head moves includes at least two first arc-shaped trajectories, and any two first arc-shaped trajectories are discontinuous. The first arc-shaped trajectory may be set according to the treatment need to meet special needs of clinical treatment plans for different treatments. For example, the first arc-shaped trajectory may be the movement trajectory which avoids the important or sensitive tissue and organ around the tumor during irradiation of radioactive rays, so that damage to normal tissues or organs during radiation therapy may be reduced. In addition, when the radiation therapy head moves along the first arc-shaped trajectory, the radiation field formed in each direction includes at least one sub-radiation field, so that the use flexibility of the radiation therapy head can be improved.

Reference is made to FIG. 3-2, which shows a flow chart of another control method for a radiation therapy device according to an embodiment of the present disclosure. The control method includes the following steps.

In step 401, a medical image of a patient is acquired.

The medical image may be a patient image formed by computed tomography (CT), nuclear magnetic resonance, positron emission tomography (PET), PET-CT or B-ultrasound. For example, the CT image of the patient can be acquired by an image guide system.

The CT image is formed according to pixels which are arranged in a matrix, and these pixels reflect a radiation absorption coefficient of the corresponding unit volume of a human body. Further, after the CT image of the patient is acquired, the CT image may be processed, for example, the CT image may be reconstructed. In a process of reconstructing the image, tissues or organs of different densities are displayed by different pseudo-colors, which enable the tumor lesion area to be positioned more accurately.

In step 402, a location of a tumor lesion area is determined according to the medical image.

In step 403, a first arc-shaped trajectory is determined according to the location of the tumor lesion area.

Further, the method may further include: a location of a designated tissue and organ around the tumor lesion area is determined according to the medical image; and the first arc-shaped trajectory is determined according to the location of the designated tissue and organ around the tumor lesion area. The designated tissue and organ may be an important or sensitive tissue and organ at risk.

In the embodiment of the present disclosure, the first arc-shaped trajectory may be determined according to the location of the tumor lesion area, or the first arc-shaped trajectory may be determined in combination with the location of the tumor lesion area and the location of the designated tissue and organ around the tumor lesion area.

In step 404, the radiation therapy head is driven to move along the first arc-shaped trajectory at a first speed, and the radiation therapy head is controlled to emit rays and form a radiation field.

The radiation field includes at least one sub-radiation field, and the first speed is smaller than a preset speed threshold. Alternatively, the radiation field formed by the radiation therapy head includes a plurality of sub-radiation fields.

For example, the preset speed threshold ranges from 2 minutes per revolution to 6 minutes per revolution. For example, the first speed is 15 minutes per revolution.

As shown in FIG. 2-2, the radiation therapy head performs a circumferential movement. A circumferential trajectory along which the radiation therapy head moves includes at least two first arc-shaped trajectories 01, and any two first arc-shaped trajectories 01 are discontinuous.

In the embodiment of the present disclosure, when the radiation therapy head moves along the first arc-shaped trajectory, the movement speed of the radiation therapy head is low in. The radiation field formed by the radiation therapy head in each direction includes a plurality of sub-radiation fields. The use flexibility of the radiation therapy head is relatively high.

In step 405, the radiation therapy head is driven to move along the second arc-shaped trajectory at a second speed, and the radiation therapy head is controlled to emit rays and form a radiation field.

The radiation field includes at least one sub-radiation field. The second speed is larger than or equal to the preset speed threshold. Alternatively, the radiation field formed by the radiation therapy head includes a plurality of sub-radiation fields. The preset speed threshold ranges from 2 minutes per revolution to 6 minutes per revolution. For example, the second speed may be 6 minutes per revolution.

In the embodiment of the present disclosure, when the radiation therapy head moves along the second arc-shaped trajectory, the movement speed of the radiation therapy head is high, which accelerates the treatment speed of the entire process and shortens the treatment time. In addition, the radiation field formed by the radiation therapy head in each direction includes a plurality of sub-radiation fields. The use reliability of the radiation therapy head is relatively high.

Further, as shown in FIG. 2-2, the circumferential trajectory along which the radiation therapy head moves further includes a second arc-shaped trajectory 02, wherein the first arc-shaped trajectory 01 and the second arc-shaped trajectory 02 are alternately arranged. The second arc-shaped trajectory may be determined according to the location of the tumor lesion area. The treatment plan may further include the second arc-shaped trajectory.

In the embodiment of the present disclosure, the first arc-shaped trajectory corresponds to the tumor lesion area, and the second arc-shaped trajectory may correspond to the designated tissue and organ around the tumor lesion area. When the radiation therapy head moves along the first arc-shaped trajectory, the movement speed of the radiation therapy head decreases, and the radiation therapy head forms the radiation field including a plurality of sub-radiation fields in each direction, so that the tumor lesion area obtains relatively high radiation dose. When the radiation therapy head moves along the second arc-shaped trajectory, the movement speed of the radiation therapy head increases, so that the important or sensitive tissue and organ at risk obtains a relatively low radiation dose. Accordingly, the damage of radiation to the normal tissue and organ is reduced.

Alternatively, the radiation field formed by the radiation therapy head includes the plurality of sub-radiation fields, and the sizes and the shapes of the plurality of sub-radiation fields are different from each other. When the radiation therapy head moves along the first arc-shaped trajectory or the second arc-shaped trajectory, the radiation field in each direction includes a plurality of sub-radiation fields, and sizes and shapes of the plurality of sub-radiation fields are different from each other. The amount of the rays can be effectively adjusted by the plurality of sub-radiation fields. Therefore, a treatment process has a relatively large space for optimization, so that the requirement for radiation dose distribution can be effectively met.

In addition, the sizes and the shapes of the plurality of sub-radiation fields included in the radiation field may be the same.

In an implementation, that the radiation therapy head is controlled to emit rays and form a radiation field includes:

the radiation therapy head is controlled to continuously emit rays and sequentially form a plurality of sub-radiation fields. The sizes and the shapes of the plurality of sub-radiation fields may be different or the same. Further, the intensity of the rays emitted from the radiation therapy head within unit time may be adjusted while the radiation therapy head is controlled to emit the rays.

In another implementation, that the radiation therapy head is controlled to emit rays and form a radiation field includes:

the radiation therapy head is controlled to emit the rays every time one sub-radiation field is formed. The sizes and the shapes of the plurality of sub-radiation fields may be different or the same. Further, the intensity of the rays emitted from the radiation therapy head within unit time may be adjusted while the radiation therapy head is controlled to emit the rays.

In step 406, the radiation therapy head is driven to move along the second arc-shaped trajectory at a second speed, and the radiation therapy head is controlled to stop emitting the rays.

Certainly, it is also possible that when the radiation therapy head moves along the second arc-shaped trajectory at the second speed, the radiation therapy head is controlled to stop emitting the rays.

When the radiation therapy head moves along the second arc-shaped trajectory at the second speed, the radiation therapy head may be controlled to stop emitting the rays. By controlling the radiation therapy head to stop emitting the rays, the amount of the rays passing through the important or sensitive tissue and organ at risk is further reduced. Accordingly, the radiation dose obtained is relatively low.

In addition, by reducing the intensity of the rays, the radiation dose obtained by the important or sensitive tissue and organ at risk may be relatively low. Particularly, in the step 405, that the radiation therapy head is driven to move along the second arc-shaped trajectory at the second speed and the radiation therapy head is controlled to emit the rays may include: the radiation therapy head is driven to move along the second arc-shaped trajectory at the second speed, and the radiation therapy head is controlled to emit the rays with an intensity less than a preset intensity threshold.

Alternatively, at least two of central angles corresponding to all the first arc-shaped frames included in the circumferential trajectory are different. Further, the central angles corresponding to all the first arc-shaped trajectories may be the same, which will be determined according to actual needs.

As shown in FIG. 2-5, a radiation therapy head 300 includes a ray source 310 and a multi-leaf collimator 320. Both the ray source and the multi-leaf collimator are connected with the control module.

Correspondingly, in the step 404 and the step 405, that the radiation therapy head is controlled to emit rays and form a radiation field includes:

the ray source is controlled to emit rays, and the multi-leaf collimator is controlled to form at least one sub-radiation field. For example, the ray source may be an accelerator or an isotope ray source.

In summary, according to the control method for the radiation therapy device according to the embodiment of the present disclosure, the control module can control the radiation therapy head to emit the rays and form the radiation field when the radiation therapy head moves along the first arc-shaped trajectory at the first speed, wherein the radiation field includes at least one sub-radiation field, and the first speed is less than the preset speed threshold. The radiation therapy head performs the circumferential movement, and the circumferential trajectory along which the radiation therapy head moves includes at least two first arc-shaped trajectories, and any two first arc-shaped trajectories are discontinuous. The radiation therapy head may avoid the important or sensitive tissue and organ when moving along the first arc-shaped trajectory, thereby preventing the important or sensitive tissue and organ from being damaged. The control method can meet the requirement for radiation dose distribution, such that a treatment plant has a relatively large space for optimization. A better treatment plant for treatment may be generated, and a larger possibility for improving the quality of the treatment plan and the treatment effect is provided. In addition, when the radiation therapy head moves along the first arc-shaped trajectory, the radiation field formed in each direction includes at least one sub-radiation field, so that the use flexibility of the radiation therapy head is improved, and a larger possibility for improving the quality of the treatment plan and the treatment effect is provided.

It should be noted that the sequence of the steps of the control method for the radiation therapy device provided by the embodiment of the present disclosure may be appropriately adjusted, and accordingly, the steps may be increased or decreased according to the circumstances. A method, variations of which are readily conceivable for those skilled in the art within the technical scope disclosed in the present disclosure, should fall within the scope of protection of the present disclosure, and therefore will be omitted again.

It should be clearly understood for those skilled in the art that for the convenience and brevity of description, the processes in the embodiments of the method may refer to corresponding processes in the embodiments of the apparatus, and will be omitted here.

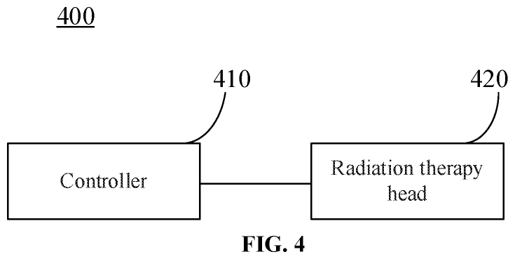

Reference is made to FIG. 4, which shows a schematic diagram showing a structure of a radiation treatment system 400 according to an embodiment of the present disclosure. The radiation treatment system 400 includes a controller 410 and a radiation therapy head 420. The controller is connected with the radiation therapy head. The controller is configured to control the radiation therapy device to move, and control the radiation therapy head to emit rays and form a radiation field.

The controller 410 includes the control apparatus for the radiation therapy device shown in FIG. 2-1.

Further, the radiation therapy system further includes a frame, wherein the controller is electrically connected with the frame, and the radiation therapy head is disposed on the frame.

The controller controls the radiation therapy head to move through the frame. The radiation therapy head can rotate with the frame.

In summary, according to the radiation treatment system provided by the embodiment of the present disclosure, the radiation therapy head can be controlled to emit the rays and form the radiation field when the radiation therapy head moves along the first arc-shaped trajectory at the first speed, wherein the radiation field includes at least one sub-radiation field, and the first speed is less than the preset speed threshold. The radiation therapy head performs the circumferential movement, and the circumferential trajectory along which the radiation therapy head moves includes at least two first arc-shaped trajectories, and any two first arc-shaped trajectories are discontinuous. The first arc-shaped trajectory can be set according to the treatment need to meet special needs of clinical treatment plans for different treatments. In addition, when the radiation therapy head moves along the first arc-shaped trajectory, the radiation field formed in each direction includes at least one sub-radiation field, so that the use flexibility of the radiation therapy head is improved, and a larger possibility for improving the quality of the treatment plan and the treatment effect is provided.

It should be noted that a type and a treatment method of the radiation therapy device will not be limited in the radiation therapy system of the present disclosure. For example, the radiation therapy device may be an accelerator or a gamma knife. The radiation therapy device may further include an imaging system, and the imaging system may include one bulb tube and one detector, or may include two bulb tubes and two detectors. Certainly, the radiation therapy system may further include an electronic portal imaging device (EPID) to verify a treatment beam. Moreover, when the radiation therapy system includes a detector, the detector may slide, thereby moving at different locations to receive a beam.

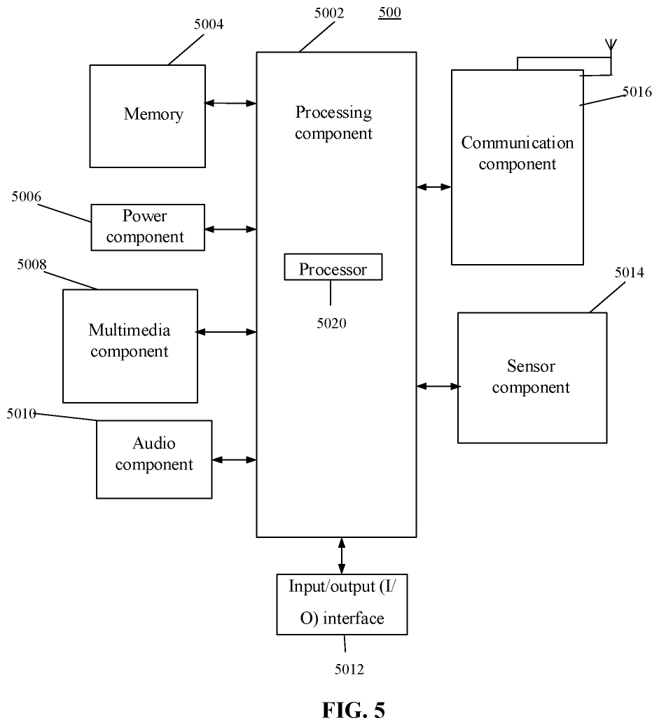

FIG. 5 is a block diagram of a control apparatus 500 of a radiotherapy device according to an embodiment. For example, the apparatus 500 may be a computer console.

Referring to FIG. 5, the apparatus 500 may include one or more of the following components: a processing component 5002, a memory 5004, a power component 5006, a multimedia component 5008, an audio component 5010, an input/output (I/O) interface 5012, a sensor component 5014, and a communication component 5016.

The processing component 5002 typically controls overall operations of the apparatus 500, such as the operations associated with display, telephone calls, data communications, camera operations, and recording operations. The processing component 5002 may include one or more processors 5020 to execute instructions to perform all or part of the steps in the above described methods. Moreover, the processing component 5002 may include one or more modules which facilitate the interaction between the processing component 5002 and other components. For instance, the processing component 5002 may include a multimedia module to facilitate the interaction between the multimedia component 5008 and the processing component 5002.

The memory 5004 is configured to store various types of data to support the operation of the apparatus 500. Examples of such data include instructions for any applications or methods operated on the apparatus 500, contact data, phonebook data, messages, pictures, video, etc. The memory 5004 may be implemented using any type of volatile or non-volatile memory devices, or a combination thereof, such as a static random access memory (SRAM), an electrically erasable programmable read-only memory (EEPROM), an erasable programmable read-only memory (EPROM), a programmable read-only memory (PROM), a read-only memory (ROM), a magnetic memory, a flash memory, a magnetic or optical disk.

The power component 5006 provides power to various components of the apparatus 500. The power component 5006 may include a power management system, one or more power sources, and any other components associated with the generation, management, and distribution of power in the apparatus 500.

The multimedia component 5008 includes a screen providing an output interface between the apparatus 500 and the user. In some embodiments, the screen may include a liquid crystal display (LCD) and a touch panel (TP). If the screen includes the touch panel, the screen may be implemented as a touch screen to receive input signals from the user. The touch panel includes one or more touch sensors to sense touches, slips, and gestures on the touch panel. The touch sensors may not only sense a boundary of a touch or slip action, but also sense a period of time and a pressure associated with the touch or slip action. In some embodiments, the multimedia component 5008 includes a front camera and/or a rear camera. The front camera and the rear camera may receive an external multimedia datum while the apparatus 500 is in an operation mode, such as a photographing mode or a video mode. Each of the front camera and the rear camera may be a fixed optical lens system or have a focal length and optical zoom capability.

The audio component 5010 is configured to output and/or input audio signals. For example, the audio component 5010 includes a microphone (MIC). When the apparatus 500 is in an operation mode, such as a call mode, a recording mode, and a voice recognition mode, the microphone is configured to receive an external audio signal. The received audio signal may be further stored in the memory 5004 or transmitted via the communication component 5016. In some embodiments, the audio component 5010 further includes a speaker to output audio signals.

The I/O interface 5012 provides an interface between the processing component 5002 and a peripheral interface module. The peripheral interface module may be a keyboard, a click wheel, buttons, and the like. The buttons may include, but are not limited to: a home button, a volume button, a starting button, and a locking button.

The sensor component 5014 includes one or more sensors to provide status assessments of various aspects of the apparatus 500. For instance, the sensor component 5014 may detect an open/closed status of the apparatus 500 and relative positioning of components, e.g., the display and the keypad of the apparatus 500. The sensor component 5014 may also detect a change in position of the apparatus 500 or a component of the apparatus 500, a presence or absence of user's contact with the apparatus 500, an orientation or an acceleration/deceleration of the apparatus 500, and a change in temperature of the apparatus 500. The sensor component 5014 may include a proximity sensor configured to detect the presence of nearby objects without any physical contact. The sensor component 5014 may also include a light sensor, such as a CMOS or CCD image sensor, for use in imaging applications. In some embodiments, the sensor component 5014 may also include an accelerometer sensor, a gyroscope sensor, a magnetic sensor, a pressure sensor, or a temperature sensor.

The communication component 5016 is configured to facilitate wired or wireless communication between the apparatus 500 and other devices. The apparatus 500 can access a wireless network based on a communication standard, such as WiFi, 2G, or 3G, or a combination thereof. In one exemplary embodiment, the communication component 5016 receives a broadcast signal or broadcast associated information from an external broadcast management system via a broadcast channel. In one exemplary embodiment, the communication component 5016 further includes a near field communication (NFC) module to facilitate short-range communications. For example, the NFC module may be implemented based on a radio frequency identification (RFID) technology, an infrared data association (IrDA) technology, an ultra-wideband (UWB) technology, a Bluetooth (BT) technology, and other technologies.

In exemplary embodiments, the apparatus 500 may be implemented with one or more application specific integrated circuits (ASICs), digital signal processors (DSPs), digital signal processing devices (DSPDs), programmable logic devices (PLDs), field programmable gate arrays (FPGAs), controllers, micro-controllers, microprocessors, or other electronic components, for performing the above methods.

In exemplary embodiments, there is also provided a non-temporary computer-readable storage medium including instructions, such as the memory 5004 including instructions. These instructions may be loaded and executed by the processor 5020 in the apparatus 500 for performing the above methods. For example, the non-transitory computer-readable storage medium may be a ROM, a RAM, a CD-ROM, a magnetic tape, a floppy disc, an optical data storage device, or the like.

A non-temporary computer-readable storage medium is provided. When the instructions in the storage medium are executed by the processor of the apparatus 500, the apparatus 500 can execute the control method of a radiotherapy device provided by the above embodiments.

The above-mentioned embodiments can be implemented in whole or in part by software, hardware, firmware, or any combination thereof. When implemented by terminal software products, these terminal software products include one or more instructions. When the instructions are loaded and executed on the terminal, the process or functions in accordance with the embodiments of the present invention can be generated in whole or in part. The terminal may include a general purpose computer, a computer network, or other programmable device. The instructions may be stored in the readable storage medium, or transferred from one readable storage medium to another readable storage medium. For example, the instructions can be transferred from a web site, a computer, a server, or a data center to another web site, computer, server, or data center, via wired network, such as coaxial cable, optical fiber, digital subscriber line (DSL), or wireless network, such as infrared, wireless, microwave. The terminal-readable storage medium may be any available medium or data storage devices including the servers and data centers integrated with one or more available mediums accessible by the terminals. The available mediums may be magnetic medium (floppy disks, hard disks, magnetic tapes, etc.), optical medium, or semiconductor medium (Solid State Disk), etc.

Persons of ordinary skill in the art can understand that all or part of the steps described in the above embodiments can be completed through hardware, or through relevant hardware instructed by applications stored in a non-transitory computer readable storage medium, such as a read-only memory, a disk or a CD, etc.

The foregoing descriptions are merely exemplary embodiments of the present disclosure, and are not intended to limit the present disclosure. Within the spirit and principles of the disclosure, any modifications, equivalent substitutions, improvements, etc., are within the protection scope of the present disclosure.

1. An apparatus for controlling a radiation therapy device, comprising a drive module, a control module and an input module, wherein,
the drive module and the control module are connected with the input module, respectively;
the input module is configured to acquire a first arc-shaped trajectory, and the first arc-shaped trajectory is determined according to a medical image of a patient;
the drive module is configured to drive a radiation therapy head to move along the first arc-shaped trajectory at a first speed, and the first speed is smaller than a preset speed threshold;
the control module is configured to control the radiation therapy head to emit rays and form a radiation field when the radiation therapy head moves along the first arc-shaped trajectory, and the radiation field comprises at least one sub-radiation field; and
the radiation therapy head performs a circumferential movement, and a circumferential trajectory along which the radiation therapy head moves comprises at least two first arc-shaped trajectories and any two first arc-shaped trajectories are discontinuous.

2. The apparatus for controlling a radiation therapy device according to claim 1, wherein,
the circumferential trajectory along which the radiation therapy head moves further comprises a second arc-shaped trajectory, and the first arc-shaped trajectory and the second arc-shaped trajectory are alternately arranged;
the drive module is further configured to drive the radiation therapy head to move along the second arc-shaped trajectory at a second speed, and the second speed is larger than or equal to the preset speed threshold;
the control module is further configured to control the radiation therapy head to emit rays and form a radiation field when the radiation therapy head moves along the second arc-shaped trajectory, and the radiation field comprises at least one sub-radiation field; or
the control module is configured to control the radiation therapy head to stop emitting rays when the radiation therapy head moves along the second arc-shaped trajectory.

3. The apparatus for controlling a radiation therapy device according to claim 1, wherein the radiation field comprises a plurality of sub-radiation fields, and
the control module is configured to control the radiation therapy head to continuously emit rays and sequentially form the plurality of sub-radiation fields; or
the control module is configured to control the radiation therapy head to emit rays every time one sub-radiation field is formed.

4. The apparatus for controlling a radiation therapy device according to claim 3, wherein
sizes and shapes of the plurality of sub-radiation fields are different from each other.

5. The apparatus for controlling a radiation therapy device according to claim 1, wherein
at least two of central angles corresponding to all the first arc-shaped trajectories comprised in the circumferential trajectory are different.

6. The apparatus for controlling a radiation therapy device according to claim 2, wherein
the control module is configured to control the radiation therapy head to emit rays with an intensity less than a preset intensity threshold when the radiation therapy head moves along the second arc-shaped trajectory.

7. The apparatus for controlling a radiation therapy device according to claim 1, wherein the input module is configured to:
acquire the medical image of the patient, and determine a location of a tumor lesion area according to the medical image; and
determine the first arc-shaped trajectory according to the location of the tumor lesion area.

8. The apparatus for controlling a radiation therapy device according to claim 7, wherein
the input module is further configured to determine a location of a designated tissue and organ around the tumor lesion area according to the medical image, and determine the first arc-shaped trajectory according to the location of the designated tissue and organ around the tumor lesion area.

9. The apparatus for controlling a radiation therapy device according to claim 1, wherein the radiation therapy head comprises a ray source and a multi-leaf collimator, and
the control module is configured to control the ray source to emit rays and control the multi-leaf collimator to form the at least one sub-radiation field.

10. A method for controlling a radiation therapy device, comprising:
acquiring a first arc-shaped trajectory, wherein the first arc-shaped trajectory is determined according to a medical image of a patient; and
driving a radiation therapy head to move along the first arc-shaped trajectory at a first speed to control the radiation therapy head to emit rays and form a radiation field, wherein the radiation field comprises at least one sub-radiation field, and the first speed is smaller than a preset speed threshold;
wherein the radiation therapy head performs a circumferential movement, and a circumferential trajectory along which the radiation therapy head moves comprises at least two first arc-shaped trajectories, and any two first arc-shaped trajectories are discontinuous.

11. The method according to claim 10, wherein the circumferential trajectory along which the radiation therapy head moves further comprises a second arc-shaped trajectory, and the first arc-shaped trajectory and the second arc-shaped trajectory are alternately arranged,
the method further comprises:
driving the radiation therapy head to move along the second arc-shaped trajectory at a second speed, and controlling the radiation therapy head to emit rays and form a radiation field or controlling the radiation therapy head to stop emitting rays, wherein the radiation field comprises at least one sub-radiation field, and the second speed is larger than or equal to the preset speed threshold.

12. The method according to claim 10, wherein the radiation field comprises a plurality of sub-radiation fields,
the controlling the radiation therapy head to emit rays and form a radiation field comprises:
controlling the radiation therapy head to continuously emit rays and sequentially form the plurality of sub-radiation fields; or
controlling the radiation therapy head to emit rays every time one sub-radiation field is formed.

13. The method according to claim 12, wherein
sizes and shapes of the plurality of sub-radiation fields are different from each other.

14. The method according to claim 10, wherein
at least two of central angles corresponding to all the first arc-shaped trajectories comprised in the circumferential trajectory are different.

15. The method according to claim 11, wherein
the driving the radiation therapy head to move along the second arc-shaped trajectory at a second speed and the controlling the radiation therapy head to emit rays comprise:
driving the radiation therapy head to move along the second arc-shaped trajectory at the second speed and controlling the radiation therapy head to emit rays with an intensity less than a preset intensity threshold.

16. The method according to claim 10, wherein
the acquiring a first arc-shaped trajectory comprises:
acquiring the medical image of the patient;
determining a location of a tumor lesion area according to the medical image; and
determining the first arc-shaped trajectory according to the location of the tumor lesion area.

17. The method according to claim 16, further comprising:
determining a location of designated tissue and organ around the tumor lesion area according to the medical image; and
determining the first arc-shaped trajectory according to the location of the designated tissue and organ around the tumor lesion area.

18. The method according to claim 10, wherein the radiation therapy head comprises a ray source and a multi-leaf collimator,
the controlling the radiation therapy head to emit rays and form a radiation field comprises:
controlling the ray source to emit rays and controlling the multi-leaf collimator to form the at least one sub-radiation field.

19. A radiation therapy system, comprising a controller and a radiation therapy head, wherein:
the controller comprises a control apparatus for a radiation therapy device according to claim 1.

20. The system according to claim 19, further comprising a frame, wherein the controller is electrically connected with the frame, the radiation therapy head is disposed on the frame and the controller controls the radiation therapy head to move through the frame.
